(19) United States
(12) Patent Application Publication
(10) Pub. No.: US 2022/0343232 A1
(43) Pub. Date: Oct. 27, 2022

(54) VEHICLE MANAGEMENT SYSTEM AND AUTONOMOUS VEHICLE

(71) Applicant: TOYOTA JIDOSHA KABUSHIKI KAISHA, Aichi-ken (JP)

(72) Inventors: Kosuke SAKAKIBARA, Aichi-ken (JP); Yoichi ONISHI, Aichi-ken (JP); Kenji KANAMORI, Aichi-ken (JP); Yusuke MATSUKAWA, Aichi-ken (JP); Kei KAWASAKI, Aichi-ken (JP); Mahito OSUKA, Aichi-ken (JP); Keisuke SAITO, Aichi-ken (JP)

(21) Appl. No.: 17/725,559

(22) Filed: Apr. 21, 2022

(30) Foreign Application Priority Data
Apr. 23, 2021 (JP) 2021-073295

(51) Int. Cl. G06Q 10/06; B60S 1/64; B60Q 1/50; B60R 25/30; B60H 1/00; G06Q 10/02; A61L 2/24

(52) U.S. Cl. CPC G06Q 10/0631; B60S 1/64; B60Q 1/544; B60Q 1/507; B60Q 1/543; B60R 25/30; B60H 1/00742; G06Q 10/02; A61L 2/24; B60W 60/0016

(57) ABSTRACT

An autonomous vehicle which transports a user through autonomous driving, having: a body temperature sensor that detects a body temperature of the user; in-cabin signage attached in a vehicle cabin; and a sanitation controller that adjusts a sanitation state in the vehicle cabin. When the body temperature of the user detected by the body temperature sensor is greater than or equal to a predetermined threshold, the sanitation controller causes the in-cabin signage to display a sanitation caution message.

### CROSS REFERENCE TO RELATED APPLICATION

This application claims priority to Japanese Patent Application No. 2021-073295 filed on Apr. 23, 2021, which is incorporated herein by reference in its entirety including the specification, claims, drawings, and abstract.

### TECHNICAL FIELD

The present disclosure relates to a structure of a vehicle management system which uses autonomous vehicles, and to a structure of an autonomous vehicle.

### BACKGROUND

In recent years, in order to prevent spreading of infectious diseases, it is desired in airplanes or the like to find, at an early stage, a user who has contracted an infectious disease with fever due to the infectious disease, and to take measures for preventing secondary infections.

For this purpose, for example, a method is proposed in which the body temperature of a passenger seated on a seat of the airplane is detected with an infrared sensor, and is displayed (for example, refer to JP 2013-39878 A).

Further, a system is proposed in which images of passengers in the airplane are captured by an infrared camera, the body temperatures of the passengers are detected based on the image, and, when a feverish patient is found, attention is called for (for example, refer to JP 2017-29219 A).

Moreover, a method is proposed in which, when a passenger in a vehicle is in a poor health condition, the poor health condition of the passenger is displayed on an outer surface of the vehicle, to notify people around the vehicle (for example, refer to JP 2020-86855 A).

In recent years, the use of autonomous vehicles for taxis and shared vehicles is being considered. In the autonomous vehicle, in many cases, no driver or crewmember embarks the vehicle, and only the user embarks the vehicle. Because of this, there may be cases in which the measure for preventing secondary infection by the driver or the crewmember checking the fever of the user as described in JP 2013-39878 A and JP 2017-29219 A may become difficult.

An advantage of the present disclosure lies in suppression of secondary infection of infectious diseases in a taxi or shared autonomous vehicle, on which no driver or crewmember embarks.

### SUMMARY

According to one aspect of the present disclosure, there is provided an autonomous vehicle which transports a user through autonomous driving, the autonomous vehicle comprising: a body temperature sensor that detects a body temperature of the user; in-cabin signage that is attached in a vehicle cabin; and a sanitation controller that adjusts a sanitation condition in the vehicle cabin, wherein, when the body temperature of the user detected by the body temperature sensor is greater than or equal to a predetermined threshold, the sanitation controller causes the in-cabin signage to display a sanitation caution message.

As described, a sanitation caution message is displayed on the in-cabin signage to notify the user of a suspicion of the infectious disease, so that the user can be encouraged to behave to prevent the secondary infection, and the spreading of the infection can be prevented.

An autonomous vehicle according to the present disclosure may further comprise a door through which the user embarks and disembarks the autonomous vehicle, and outside-vehicle signage attached outside the vehicle, and, when the body temperature of the user detected by the body temperature sensor is greater than or equal to a predetermined threshold, the sanitation controller may lock the door and cause the outside-vehicle signage to display that the vehicle is out of service, after the user disembarks the autonomous vehicle.

With this configuration, it is possible to suppress embarkment of another user onto the autonomous vehicle on which the user suspected of having the infectious disease has embarked, and to suppress occurrence of secondary infection.

An autonomous vehicle according to the present disclosure may further comprise a window that can be opened and closed, and an air-conditioning apparatus that can introduce outside air, and, when the body temperature of the user detected by the body temperature sensor is greater than or equal to a predetermined threshold, the sanitation controller may open the window and introduce the outside air with the air-conditioning apparatus to ventilate the vehicle cabin, after the user disembarks the autonomous vehicle.

With this configuration, even when another user embarks the autonomous vehicle on which the user suspected of having the infectious disease had embarked, the possibility of occurrence of secondary infection can be reduced.

An autonomous vehicle according to the present disclosure may further comprise a sterilization apparatus that sterilizes the vehicle cabin, and, when the body temperature of the user detected by the body temperature sensor is greater than or equal to a predetermined threshold, the sanitation controller may actuate the sterilization apparatus and execute sterilization of the vehicle cabin, after the user disembarks the autonomous vehicle.

With this configuration, even after embarkment of a user suspected of having the infectious disease, another user can embark the autonomous vehicle after the sterilization.

According to one aspect of the present disclosure, there is provided a vehicle management system comprising: a plurality of autonomous vehicles that transport users through autonomous driving; and a management supervision server that supervises management of the plurality of autonomous vehicles, wherein each of the autonomous vehicles comprises: a body temperature sensor which detects a body temperature of the user; and a sanitation controller which adjusts a sanitation condition in the vehicle cabin based on information from the body temperature sensor, and, when the body temperature of the user detected by the body temperature sensor is greater than or equal to a predetermined threshold, the sanitation controller transmits user body temperature information to the management supervision server.

As described above, the user body temperature information is transmitted to the management supervision server when the body temperature of the user is greater than or equal to a threshold. Thus, an autonomous vehicle can be appropriately managed after embarkment of a user suspected of having the infectious disease.

In a vehicle management system of the present disclosure, when the management supervision server receives the user body temperature information from the sanitation controller, the management supervision server may: identify the one of the autonomous vehicles that has transmitted the user body temperature information as a necessary-to-isolate vehicle; cancel an embarking reservation for the necessary-to-isolate vehicle after the reception of the user body temperature information; and dispatch another one of the autonomous vehicles, different from the necessary-to-isolate vehicle.

A vehicle management system according to the present disclosure may further comprise a vehicle station that has a parking area for a plurality of the autonomous vehicles, and in which maintenance and inspection are performed for the plurality of autonomous vehicles, and, when the management supervision server receives the user body temperature information from the sanitation controller, the management supervision server may: identify the one of the autonomous vehicles that has transmitted the user body temperature information as a necessary-to-isolate vehicle; and transmit, to the necessary-to-isolate vehicle, a service command for returning to the vehicle station. With this configuration, it becomes possible to suppress occurrence of secondary infection by another user embarking the autonomous vehicle on which the user suspected of having the infectious disease had embarked.

The vehicle station may comprise a sterilization apparatus which executes sterilization of the vehicle cabin of the autonomous vehicle, and a station terminal which communicates with the management supervision server, and the management supervision server may transmit, to the station terminal, a message indicating that the necessary-to-isolate vehicle must be moved to the sterilization apparatus of the vehicle station, and a sterilization process must be executed for the necessary-to-isolate vehicle.

With this configuration, it becomes possible to urge a worker in the vehicle station to perform the sterilization process of the necessary-to-isolate vehicle, and to suppress secondary infection of the worker in the vehicle station.

The present disclosure can suppress secondary infection of the infectious disease in taxis and shared vehicles which use autonomous vehicles on which no driver or crewmember embarks.

### BRIEF DESCRIPTION OF DRAWINGS

Embodiment(s) of the present disclosure will be described based on the following figures, wherein:

FIG. 1 is a schematic system diagram showing a structure of a vehicle management system according to an embodiment of the present disclosure;

FIG. 2 is a diagram showing a data structure of an operation state database shown in FIG. 1;

FIG. 3 is a diagram showing a data structure of a user database shown in FIG. 1;

FIG. 4 is a system diagram showing a structure of a vehicle used in the vehicle management system according to the embodiment of the present disclosure;

FIG. 5 is a control block diagram of the vehicle shown in FIG. 4;

FIG. 6 is a flowchart showing an operation of a management supervision server used in the vehicle management system according to the embodiment of the present disclosure;

FIG. 7 is a flowchart showing an operation of a sanitation controller of a vehicle used in the vehicle management system according to the embodiment of the present disclosure;

FIG. 8 is a flowchart showing another operation of the sanitation controller of the vehicle used in the vehicle management system according to the embodiment of the present disclosure;

FIG. 9 is a flowchart showing another operation of the management supervision server of the vehicle management system according to the embodiment of the present disclosure; and

FIG. 10 is a flowchart showing an operation of a travel controller of the vehicle used in the vehicle management system according to the embodiment of the present disclosure.

### DESCRIPTION OF EMBODIMENTS

A vehicle management system 100, and a vehicle 10 which can be autonomously driven and is managed by the vehicle management system 100 will now be described with reference to the drawings.

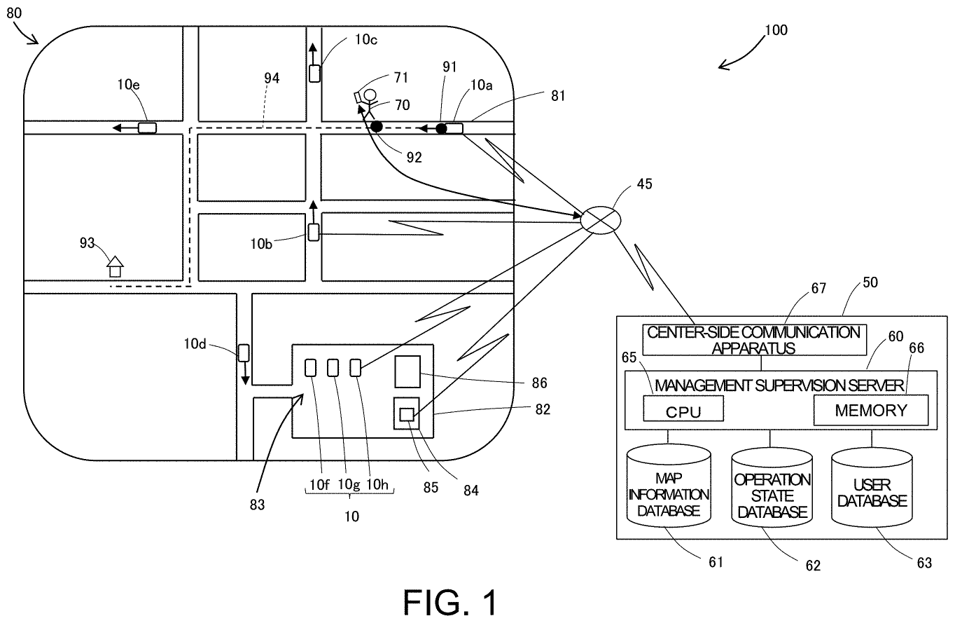

As shown in FIG. 1, the vehicle management system 100 according to the present embodiment is a system which manages a plurality of vehicles 10 which can be autonomously driven within a predetermined management area 80. The plurality of vehicles 10 are autonomous taxis which travel within the management area 80 through autonomous driving based on a command from a management supervision server 60 placed in a management supervision center 50, and transport users 70. The management supervision server 60 and the vehicle 10 are connected to each other via a wireless communication line 45 and a center-side communication apparatus 67.

In the vehicle management system 100 of the present embodiment, a configuration is described in which eight vehicles 10 are managed. When the eight vehicles 10 are to be distinguished, the vehicles will be described as vehicles 10a˜10h. On the other hand, when the eight vehicles are not distinguished from each other, the vehicles will be described as the vehicles 10. FIG. 1 shows a state in which three vehicles 10f˜10h are parked in a parking area 83 of a vehicle station 82 in the management area 80, and the five remaining vehicles 10a˜10e travel on roads 81 in the management area 80. In addition, as shown in FIG. 1, in the vehicle station 82, a building 84 in which maintenance and inspection of the vehicles 10 are performed is provided, and a station terminal 85 which communicates with the management supervision server 60 and displays an operation state or the like of each vehicle is provided in the building 84. In addition, in the vehicle station 82, a sterilization apparatus 86 which performs a sterilization process of a vehicle cabin 10R of the vehicle 10 is provided. In FIG. 1, while the connection states respectively between the vehicles 10a, 10b, and 10h and the management supervision server 60 are shown, the connection states between the other vehicles 10 and the management supervision server 60 are not shown. The management supervision server 60 is a computer formed from a CPU 65 which executes information processing therein, and a memory 66 which stores an operation program, operation data, a database, or the like. A map information database 61, an operation state database 62, and a user database 63 are connected to the management supervision server 60. The map information database 61 is a database which stores map information.

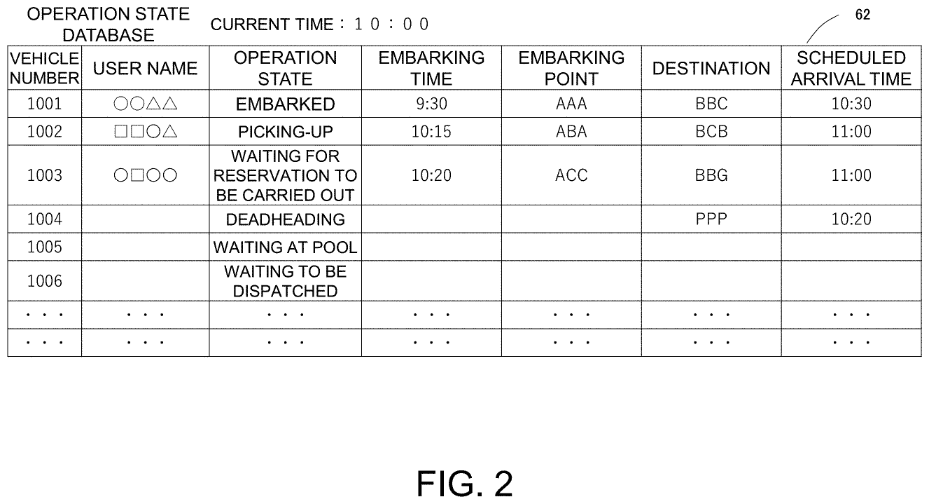

As shown in FIG. 2, the operation state database 62 is a database which stores vehicle numbers of the plurality of vehicles 10, whose management is supervised by the management supervision server 60, a current operation state, a name of the user 70, an embarking time when the user 70 embarks, an embarking point, a destination, and a scheduled arrival time, in correlation with each other. Here, as the operation state, the operation state of each vehicle 10 is stored, such as embarked, picking-up, waiting for reservation to be carried out, deadheading, waiting at a pool, waiting to be dispatched, and the like. “Embarked” indicates that the user 70 has embarked the vehicle 10, and the vehicle is moving toward the destination. “Picking-up” indicates that the vehicle is moving toward a pick-up point, after being requested by the user 70. “Waiting for reservation to be carried out” indicates that the vehicle is dispatched after accepting a reservation by the user 70, and is waiting until a use start time. “Deadheading” indicates that the use by the user 70 has completed, and the vehicle is on the way to return to the vehicle station 82. “Waiting at a pool” indicates that charging of the vehicle is completed, and the vehicle is waiting in the vehicle station 82 in an operable state. “Waiting to be dispatched” indicates that one round of service is completed, and the vehicle is waiting for a next dispatch. For the embarking time, the time when the user 70 has embarked the vehicle is stored if the user 70 has embarked the vehicle at a time prior to the current time, or a scheduled embarking time is stored if the user 70 is scheduled to embark at a time later than the current time. Similarly, for the embarking point, a point where the user has actually embarked the vehicle or a scheduled embarking point is stored. When there is no scheduled embarkment, the field is left blank.

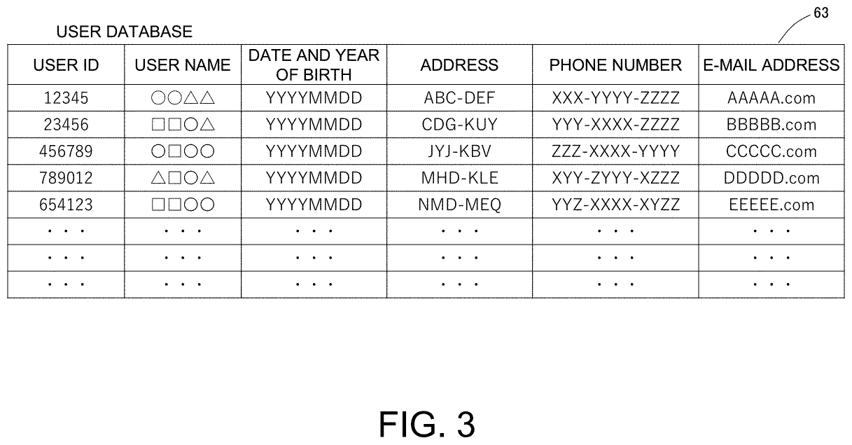

As shown in FIG. 3, the user database 63 is a database which stores a user ID, a name, a date and year of birth, an address, a phone number, and an e-mail address of the user 70 who uses the vehicle 10, in correlation with each other.

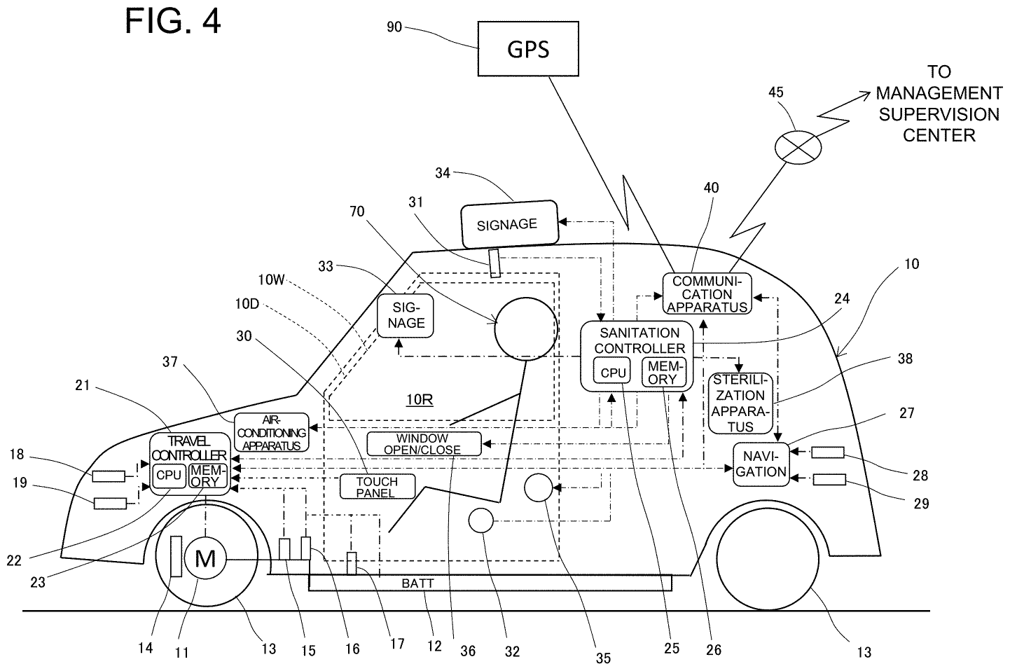

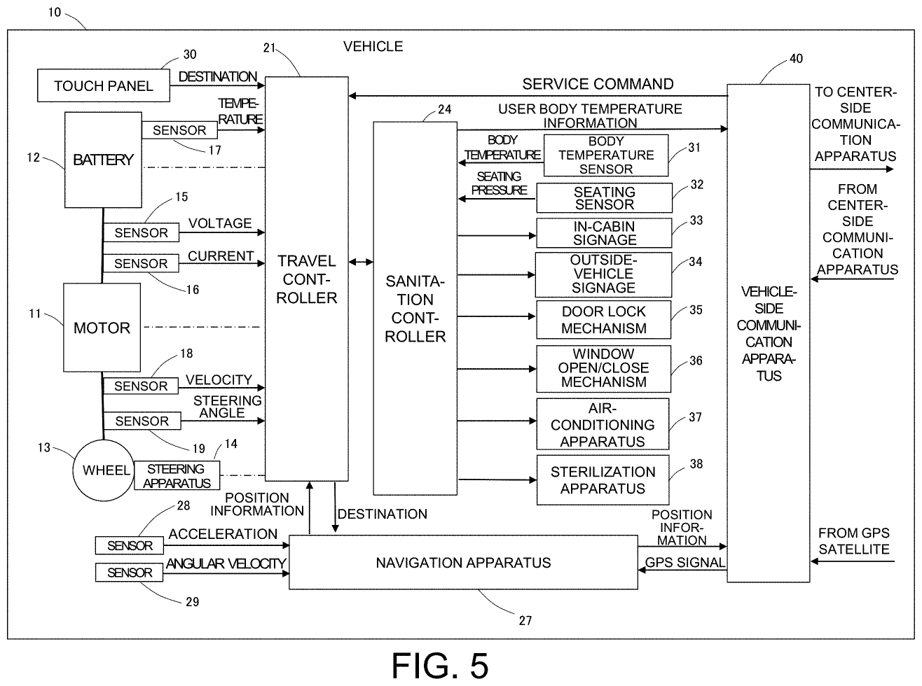

As shown in FIGS. 4 and 5, the vehicle 10 is an Battery Electric vehicle which can be autonomously driven, and comprises a driving motor 11, a battery 12 which supplies driving power to the motor 11, a steering apparatus 14 which adjusts a steering angle of a wheel 13, a travel controller 21 which controls traveling of the vehicle, a sanitation controller 24 which adjusts a sanitation condition in a vehicle cabin, a navigation apparatus 27, and a vehicle-side communication apparatus 40.

The motor 11, the battery 12, and the steering apparatus 14 are connected to the travel controller 21. Further, a voltage sensor 15 and a current sensor 16 for respectively detecting a voltage and a current of the power supplied from the battery 12 to the motor 11 are connected between the battery 12 and the motor 11. Moreover, a temperature sensor 17 for detecting a temperature of the battery 12 is connected to the battery 12. In addition, a vehicle velocity sensor 18 for detecting a vehicle velocity, and a steering angle sensor 19 for detecting the steering angle of the wheel 13 are attached between the motor 11 and the wheel 13. The voltage sensor 15, the current sensor 16, the temperature sensor 17, the vehicle velocity sensor, and the steering angle sensor 19 are connected to the travel controller 21.

An acceleration sensor 28 and an angular velocity sensor 29 respectively for detecting an acceleration and an angular velocity of the vehicle 10 are attached to the vehicle 10. The acceleration sensor 28 and the angular velocity sensor 29 are connected to the navigation apparatus 27. The navigation apparatus 27 identifies a current position of the vehicle 10 based on a GPS signal acquired from a GPS satellite 90 via the vehicle-side communication apparatus 40, and the acceleration information and the angular velocity information of the vehicle 10 acquired from the acceleration sensor 28 and the angular velocity sensor 29, and outputs position information of the vehicle 10 to the travel controller 21. Further, the navigation apparatus 27 outputs current position information of the vehicle 10 to the management supervision center 50 via the wireless communication line 45. In addition, the vehicle 10 includes a touch panel 30 for the user 70 to input a destination 93.

The travel controller 21 is a computer having a CPU 22 which executes an information process therein, and a memory 23 which stores software and programs executed by the CPU 22, and data. The travel controller 21 executes, with the CPU 22, software stored in the memory 23 based on a service command which is input from the management supervision center 50, or the destination 93 which is input from the touch panel 30, the current position information which is input from the navigation apparatus 27, and the input data from various sensors 15˜19, to control the motor 11, the battery 12, and the steering apparatus 14, to thereby cause the vehicle 10 to autonomously travel.

On the vehicle 10, a door 10D for the user 70 to embark or disembark the vehicle cabin 10R is provided. A window 10W which can be opened and closed is provided on the door 10D. A door lock mechanism 35 for fixing opening/closing of the door 10D, and a window open/close mechanism 36 for opening/closing the window 10W are attached to the door 10D. In addition, at a front part of the vehicle 10, an air-conditioning apparatus 37 for executing the air conditioning in the vehicle cabin 10R and introducing outside air to the vehicle cabin 10R is equipped. At a rear part of the vehicle 10, a sterilization apparatus 38 for sterilizing the inside of the vehicle cabin is equipped. Further, a body temperature sensor 31, a seating sensor 32, and in-cabin signage 33 are attached in the vehicle cabin 10R. On a roof of the vehicle 10, outside-vehicle signage 34 is attached.

The body temperature sensor 31 is attached on a ceiling of the vehicle cabin 10R, and detects a body temperature of the user 70 seated on a seat (not shown). The body temperature sensor 31 may be, for example, an infrared type thermometer. The in-cabin signage 33 may be any apparatus that allows visible recognition of a message by the user 70 seated on the seat. The in-cabin signage 33 may be, for example, a display attached to an instrument panel, or a head-up display which projects a message on a windshield. In the vehicle 10 of the present embodiment, the outside-vehicle signage 34 is a display attached on the roof, but is not limited to such a configuration, and, for example, so long as the message can be recognized from outside of the vehicle, a configuration may be employed in which a message can be displayed on the window 10W, to thereby form the outside-vehicle signage 34. Further, on the seat (not shown), the seating sensor 32 for detecting whether or not the user 70 is seated is attached.

The body temperature sensor 31 and the seating sensor 32 are connected to the sanitation controller 24, and the body temperature information of the user 70 detected by the body temperature sensor 31 and the seating information are input to the sanitation controller 24. The in-cabin signage 33, the outside-vehicle signage 34, the door lock mechanism 35, the window open/close mechanism 36, the air-conditioning apparatus 37, and the sterilization apparatus 38 are connected to the sanitation controller 24, and are operated by commands of the sanitation controller 24.

The sanitation controller 24 is a computer having a CPU 25 which executes an information process therein, and a memory 26 which stores software and programs executed by the CPU 25, and data. As will be described later, based on the body temperature of the user 70 which is input from the body temperature sensor 31, the sanitation controller 24 outputs messages on the in-cabin signage 33 and the outside-vehicle signage 34, locks the door 10D with the door lock mechanism 35, opens or closes the window 10W with the window open/close mechanism 36, and actuates the air-conditioning apparatus 37 and the sterilization apparatus 38. In addition, the sanitation controller 24 is connected to the vehicle-side communication apparatus 40, and transmits the user body temperature information detected by the body temperature sensor 31 to the management supervision server 60 via the vehicle-side communication apparatus 40. Further, the sanitation controller 24 is connected to the travel controller 21, and exchanges data with the travel controller 21.

In the above, a configuration is described in which the travel controller 21 and the sanitation controller 24 are separate computers respectively having the CPUs 22 and 25 and the memories 23 and 26, but alternatively, the travel controller 21 and the sanitation controller 24 may be formed as two control blocks of one computer having a common CPU and a common memory.

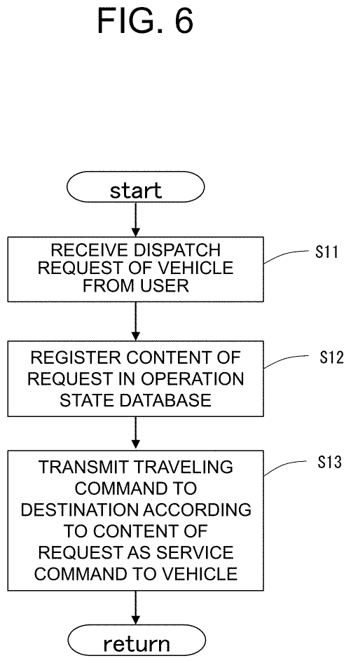

Next, with reference to FIGS. 6 to 10, an operation of the vehicle management system 100 of the present embodiment will be described. First, with reference to FIG. 6, basic operations of the management supervision server 60 and the vehicle 10 will be described.

The user 70 in the management area 80 connects a portable terminal 71 of the user 70 to the management supervision server 60, inputs the ID of the user 70, the e-mail address, the destination 93 (refer to FIG. 1), dispatch time, and the like, and requests dispatching of the vehicle 10. As shown in step S11 of FIG. 6, when the management supervision server 60 receives the dispatch request from the portable terminal 71 of the user 70, the management supervision server 60 checks whether or not the user 70 is a registered user 70, by referring to the user database 63. After successful confirmation, the process proceeds to step S12 of FIG. 6, and the management supervision server 60 registers contents of the dispatch request of the user 70 in the operation state database 62. The process proceeds to step S13 of FIG. 6, where the management supervision server 60 transmits a travel command to the destination 93 according to the request, as a service command, to the vehicle 10a. The service command includes the user name, an embarking point 92, the scheduled embarking time, and the destination 93 (refer to FIG. 1).

When the vehicle 10a receives the service command, the vehicle 10a inputs the scheduled embarking time, the embarking point 92, and the destination 93 to the navigation apparatus 27, and acquires, from the navigation apparatus 27, a travel path 94 to the destination 93 via the embarking point 92. The vehicle 10a starts the travel from a current point 91 to the embarking point 92 along the travel path 94 by the autonomous driving. When the travel to the embarking point 92 is started by the vehicle 10, the vehicle 10 transmits to the management supervision server 60 a signal indicating that the operation state is changed to picking-up. The management supervision server 60 registers the received operation state in the operation state database 62. When the vehicle 10 arrives at the embarking point 92 and the user 70 embarks the vehicle 10a, the vehicle 10 transmits, to the management supervision server 60, a signal indicating that the operation state is changed to the embarked, and an arrival time to the destination 93. The management supervision server 60 registers the received operation state in the operation state database 62. When the vehicle 10a travels along the travel path 94 and arrives at the destination 93, and the user 70 disembarks, the vehicle 10a transmits, to the management supervision server 60, a signal indicating that the service reserved by the user 70 has been completed, and the vehicle is now in the state of waiting to be dispatched. The management supervision server 60 stores the received operation state in the operation state database 62.

In the above, a configuration is described in which the user 70 inputs the destination 93 when requesting the dispatch, but alternatively, a configuration may be employed in which the user 70 inputs the destination 93 from the touch panel 30 after the user 70 embarks the vehicle 10a.

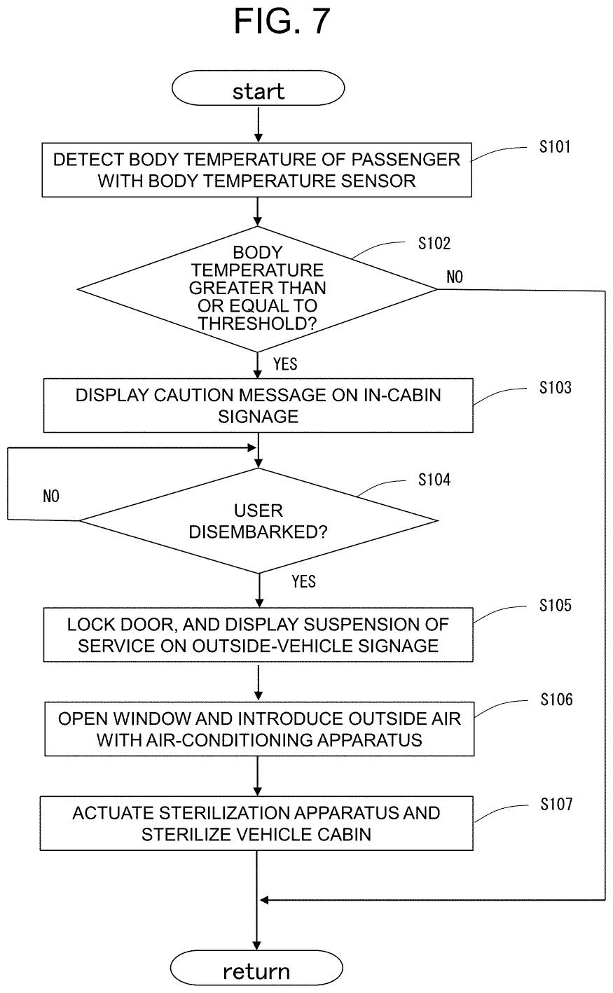

Next, with reference to FIG. 7, an operation of the sanitation controller 24 when the user 70 embarks the vehicle 10a will be described.

The sanitation controller 24 detects seating of the user 70 with the seating sensor 32 when the user 70 embarks the vehicle 10a and is seated on the seat in the vehicle cabin 10R. When the sanitation controller detects the seating of the user 70, as shown in step

S101 of FIG. 7, the sanitation controller 24 detects the body temperature of the user 70 with the body temperature sensor 31. Then, in step S102 of FIG. 7, the sanitation controller 24 judges whether or not the body temperature of the user 70 is greater than or equal to a predetermined threshold. The predetermined threshold may be set, for example, at a temperature of about 37.5° C.

When the sanitation controller 24 judges YES in step S102 of FIG. 7, the process proceeds to step S103 of FIG. 7, and the sanitation controller 24 causes the in-cabin signage 33 to display a sanitation caution message for the user 70. The sanitation caution message is a message that allows the user 70 to recognize that there is a suspicion of the infectious disease, and a possibility of causing secondary infection, such as, for example, “your body temperature is ◯◯° C. You may have an infectious disease”, “your body temperature is ◯◯° C. You may have an infectious disease. Please be diagnosed at a medical institution”, or the like. At this point, if the user 70 changes, with the touch panel 30, the destination 93 to a nearby medical institution, the vehicle 10a re-calculates the travel path 94, and travels toward the nearby medical institution. If the user 70 continues to embark without taking any action, the vehicle starts the autonomous travel toward the original destination 93.

When the vehicle 10a arrives at the destination 93 and confirms that the user 70 has disembarked, with the seating sensor 32 in step S104 of FIG. 7, the process proceeds to step S105 of FIG. 7, and the door lock mechanism 35 of the door 10D is operated to lock the door 10D, to prevent opening of the door 10D from the outside of the vehicle. A message such as “service suspended” is then displayed on the outside-vehicle signage 34. In this manner, it is possible to suppress embarking of another user on the vehicle 10a on which the user 70 suspected of having the infectious disease had embarked, and consequently, occurrence of the secondary infection.

In step S106 of FIG. 7, the sanitation controller 24 actuates the window open/close mechanism 36, and activates the air-conditioning apparatus 37 in the outside-air introduction mode, to introduce the outside air into the vehicle cabin 10R and to ventilate the vehicle cabin 10R. With this process, viruses in the vehicle cabin 10R can be discharged to the outside, and, even when another user embarks the vehicle 10a on which the user 70 suspected of having the infectious disease had embarked, the possibility of occurrence of the secondary infection can be reduced. The sanitation controller 24 also activates the equipped sterilization apparatus 38 in step S107 of FIG. 7, to sterilize the air in the vehicle cabin 10R. In this manner, even after the embarkment of the user 70 suspected of having the infectious disease, another user may be embarked on the vehicle 10a after the sterilization.

When the sanitation controller 24 judges NO in step S102 of FIG. 7, the sanitation controller 24 completes the operation.

As described, in the vehicle 10 according to the present embodiment, when the vehicle 10 is managed as an autonomously driven taxi managed in autonomous driving and on which no driver or crewmember embarks, secondary infection with an infectious disease can be suppressed.

In the above, the sanitation controller 24 is described as sequentially executing the operations of steps S105˜S107 of FIG. 7 after the passenger disembarks, but the present embodiment is not limited to such a configuration, and a configuration may be employed in which only one operation or two operations among the steps S105˜S107 of FIG. 7 are executed.

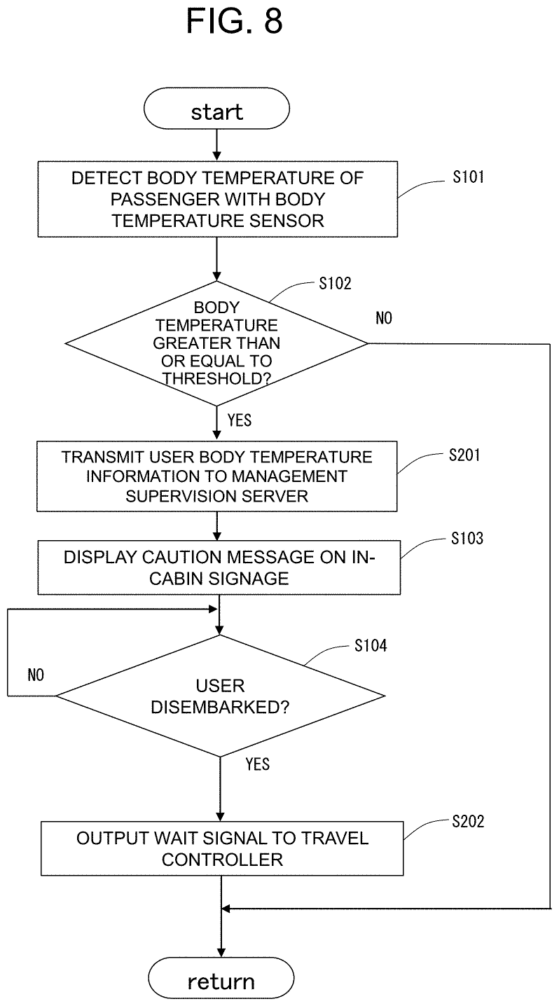

Next, with reference to FIGS. 8 to 10, an operation of the vehicle management system 100 will be described. Operations similar to those described above with reference to FIG. 7 will be assigned the same reference numerals, and will be described only briefly.

In step S101 of FIG. 8, the sanitation controller 24 of the vehicle 10 detects the body temperature of the user 70. When in step S102 the detected body temperature is found to be greater than or equal to the predetermined threshold, the process proceeds to step S201 of FIG. 8, and the sanitation controller 24 transmits the user body temperature information to the management supervision server 60. The process proceeds to step S103 of FIG. 8, and the sanitation controller 24 causes the in-vehicle signage 33 to display the sanitation caution message.

When the sanitation controller 24 confirms disembarkment of the user 70 in step S104 of FIG. 8, in step S202 of FIG. 8, the sanitation controller 24 outputs, to the travel controller 21, a wait signal indicating a state of waiting for reception of a service command from the management supervision server 60.

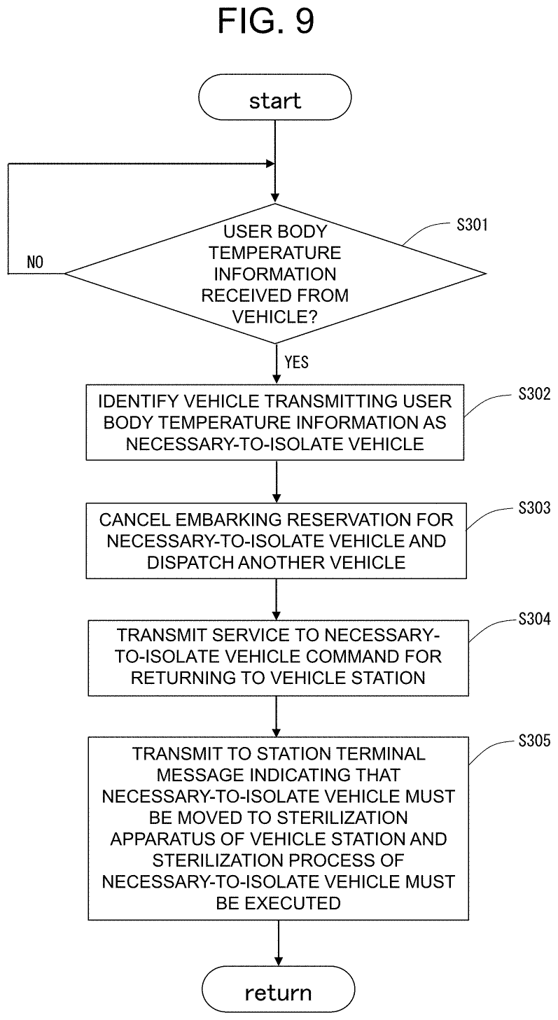

As shown in step S301 of FIG. 9, the management supervision server 60 waits until the management supervision server 60 receives the user body temperature information from the sanitation controller 24 of the vehicle 10a. When the management supervision server 60 receives the user body temperature information from the vehicle 10a, the process proceeds to step S302 of FIG. 9, and the management supervision server 60 identifies the vehicle 10a as a necessary-to-isolate vehicle. The management supervision server 60 registers data of “isolated” in the field of the operation state of the operation state database 62 shown in FIG. 2. For example, when the vehicle number of the vehicle 10a is 1001, the management supervision server 60 changes the operation state of the vehicle number of 1001 in the operation state database 62 from “waiting to be dispatched” to “isolated”.

The process proceeds to S303 of FIG. 9, and, when an embarking reservation is registered for the vehicle 10a (having the vehicle number of 1001) in the operation state database 62, the management supervision server 60 cancels the reservation, and dispatches another vehicle 10. For example, the management supervision server 60 registers the user name, the embarking time, or the like for a vehicle of “waiting to be dispatched” such as the vehicle of the vehicle number of 1006 shown in FIG. 2, and changes the operation state to “waiting for reservation to be carried out”.

The process proceeds to step S304 of FIG. 9, and the management supervision server 60 transmits to the vehicle 10a which is the necessary-to-isolate vehicle a service command for returning to the vehicle station 82.

In step S305 of FIG. 9, when the vehicle 10a which is the necessary-to-isolate vehicle arrives at the vehicle station 82, the management supervision server 60 transmits to the station terminal 85 a signal indicating that the vehicle 10a must be moved from the parking area 83 to the sterilization apparatus 86, and that the sterilization process of the vehicle cabin 10R of the vehicle 10a must be executed.

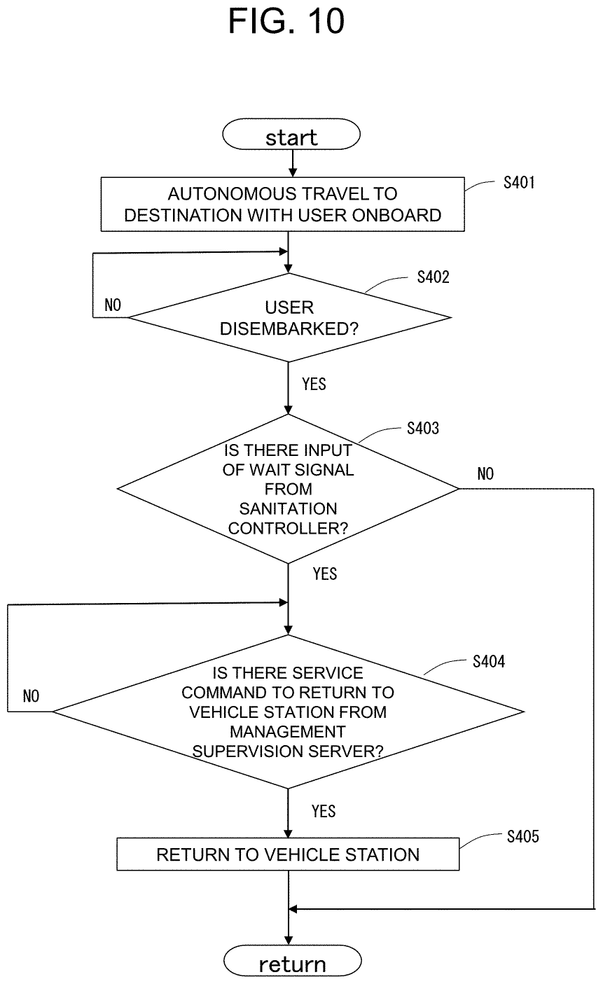

As shown in step S401 of FIG. 10, the travel controller 21 causes autonomous travel to the destination with the user 70 onboard. After the vehicle arrives at the destination, as shown in step S402 of FIG. 10, the vehicle waits until the user 70 disembarks, and, when the user disembarks, the process proceeds to step S403 of FIG. 10, and the travel controller 21 judges whether or not a wait signal is input from the sanitation controller 24. When the travel controller 21 judges YES in step S403 of FIG. 10, the process proceeds to step S404 of FIG. 10, and the travel controller 21 waits until a service command for returning to the vehicle station 82 is received from the management supervision server 60. When the service command is received, in step S404 of FIG. 10, the travel controller 21 judges YES, the process proceeds to step S405 of FIG. 10, and the vehicle starts to travel toward the vehicle station 82. When the travel controller 21 judges NO in step S403 of FIG. 10, the travel controller 21 completes the operation.

When the vehicle 10a which is the necessary-to-isolate vehicle parks in the parking area 83 of the vehicle station 82, a worker of the vehicle station 82 wears a protective suit, manually moves the vehicle 10a to the sterilization apparatus 86, and executes the sterilization process of the vehicle cabin 10R.

As described, because the sterilization of the vehicle cabin 10R is executed at the vehicle station 82, the sterilization of the vehicle cabin 10R can be effectively executed even for a case of the vehicle 10 having no sterilization apparatus 38, and the vehicle 10 can be managed again. Further, because the management supervision server 60 transmits, to the station terminal 85 and in advance, the necessity of execution of the sterilization process of the vehicle cabin 10R of the necessary-to-isolate vehicle, it is possible to suppress secondary infection due to the worker of the vehicle station 82 entering the vehicle 10 without the protective suit.

As described, in the vehicle management system 100 of the present embodiment, when the vehicle 10 is managed as an autonomous taxi managed in autonomous driving and on which no driver or crewmember embarks, secondary infection with the infectious disease can be suppressed.

In the above, the vehicle management system 100 is described as managing the vehicles 10 which can be autonomously driven as autonomous taxis, but the present embodiment is not limited to such a configuration, and, for example, the vehicles 10 may be managed as shared vehicles.

1. An autonomous vehicle which transports a user through autonomous driving, the autonomous vehicle comprising:
a body temperature sensor that detects a body temperature of the user;
in-cabin signage that is attached in a vehicle cabin; and
a sanitation controller that adjusts a sanitation condition in the vehicle cabin, wherein
when the body temperature of the user detected by the body temperature sensor is greater than or equal to a predetermined threshold, the sanitation controller causes the in-cabin signage to display a sanitation caution message.

2. The autonomous vehicle according to claim 1, further comprising:
a door through which the user embarks and disembarks the autonomous vehicle; and
outside-vehicle signage attached outside the vehicle, wherein
when the body temperature of the user detected by the body temperature sensor is greater than or equal to a predetermined threshold, the sanitation controller locks the door and causes the outside-vehicle signage to display that the vehicle is out of service, after the user disembarks the autonomous vehicle.

3. The autonomous vehicle according to claim 1, further comprising:
a window that can be opened and closed; and
an air-conditioning apparatus that can introduce outside air, wherein
when the body temperature of the user detected by the body temperature sensor is greater than or equal to a predetermined threshold, the sanitation controller opens the window and introduces the outside air with the air-conditioning apparatus to ventilate the vehicle cabin, after the user disembarks the autonomous vehicle.

4. The autonomous vehicle according to claim 2, further comprising:
a window that can be opened and closed; and
an air-conditioning apparatus that can introduce outside air, wherein
when the body temperature of the user detected by the body temperature sensor is greater than or equal to a predetermined threshold, the sanitation controller opens the window and introduces the outside air with the air-conditioning apparatus to ventilate the vehicle cabin, after the user disembarks the autonomous vehicle.

5. The autonomous vehicle according to claim 1, further comprising:
a sterilization apparatus that sterilizes the vehicle cabin, wherein
when the body temperature of the user detected by the body temperature sensor is greater than or equal to a predetermined threshold, the sanitation controller actuates the sterilization apparatus and executes sterilization of the vehicle cabin, after the user disembarks the autonomous vehicle.

6. The autonomous vehicle according to claim 2, further comprising:
a sterilization apparatus that sterilizes the vehicle cabin, wherein
when the body temperature of the user detected by the body temperature sensor is greater than or equal to a predetermined threshold, the sanitation controller actuates the sterilization apparatus and executes sterilization of the vehicle cabin, after the user disembarks the autonomous vehicle.

7. The autonomous vehicle according to claim 3, further comprising:
a sterilization apparatus that sterilizes the vehicle cabin, wherein
when the body temperature of the user detected by the body temperature sensor is greater than or equal to a predetermined threshold, the sanitation controller actuates the sterilization apparatus and executes sterilization of the vehicle cabin, after the user disembarks the autonomous vehicle.

8. The autonomous vehicle according to claim 4, further comprising:
a sterilization apparatus that sterilizes the vehicle cabin, wherein
when the body temperature of the user detected by the body temperature sensor is greater than or equal to a predetermined threshold, the sanitation controller actuates the sterilization apparatus and executes sterilization of the vehicle cabin, after the user disembarks the autonomous vehicle.

9. A vehicle management system comprising:
a plurality of autonomous vehicles that transport users through autonomous driving; and
a management supervision server that supervises management of the plurality of autonomous vehicles, wherein
each of the autonomous vehicles comprises: a body temperature sensor which detects a body temperature of the user; and a sanitation controller which adjusts a sanitation state in the vehicle cabin based on information from the body temperature sensor, and
when the body temperature of the user detected by each body temperature sensor is greater than or equal to a predetermined threshold, the sanitation controller transmits user body temperature information to the management supervision server.

10. The vehicle management system according to claim 9, wherein
when the management supervision server receives the user body temperature information from the sanitation controller, the management supervision server:
identifies the one of the autonomous vehicles which has transmitted the user body temperature information as a necessary-to-isolate vehicle;
cancels an embarking reservation for the necessary-to-isolate vehicle after the reception of the user body temperature information; and
dispatches another one of the autonomous vehicles, different from the necessary-to-isolate vehicle.

11. The vehicle management system according to claim 9, further comprising:
a vehicle station that has a parking area for a plurality of the autonomous vehicles, and in which maintenance and inspection are performed for the plurality of autonomous vehicles, wherein
when the management supervision server receives the user body temperature information from the sanitation controller, the management supervision server:
identifies the one of the autonomous vehicles which has transmitted the user body temperature information as a necessary-to-isolate vehicle; and
transmits, to the necessary-to-isolate vehicle, a service command for returning to the vehicle station.

12. The vehicle management system according to claim 11, wherein the vehicle station comprises:
a sterilization apparatus which executes sterilization of the vehicle cabin of the autonomous vehicle; and
a station terminal which communicates with the management supervision server, and
the management supervision server transmits, to the station terminal, a message indicating that the necessary-to-isolate vehicle must be moved to the sterilization apparatus of the vehicle station, and a sterilization process must be executed for the necessary-to-isolate vehicle.
